(19) United States
(12) Patent Application Publication
(10) Pub. No.: US 2012/0301405 A1
(43) Pub. Date: Nov. 29, 2012

(54) Simple Mechanical Procedure and Product for Deterring Substance Abuse.

(76) Inventor: John C. Kulli, Upper St. Clair, PA (US)

(21) Appl. No.: 13/374,844

(22) Filed: Jan. 18, 2012

Related U.S. Application Data
(63) Continuation-in-part of application No. 11/807,355, filed on May 25, 2007.
Application No. 11/807,355 is a continuation of application No. 11/406,886, filed on Apr. 19, 2006, now abandoned.
(60) Provisional application No. 60/676,865, filed on May 2, 2005.

(51) Int. Cl. A61K 31/5513

(52) U.S. Cl. 424/10.1; 514/221

(57) ABSTRACT

A drug-formulating method, a drug commercial-distribution method, and a drug formulation improve safety of a drug that is at risk for abuse—such as methylphenidate, or amphetamine, or an amphetamine-like central-nervous-system stimulant, and particularly a benzodiazepine. The drug is formulated into a form (not a transdermal patch) that tends to deter conversion to powder; and in this form commercially distributed—preferably enclosed in, or dissolved or dispersed into or onto, a nontoxic carrier such as a capsule, for example a gel, e.g., methylcellulose, hydroxymethylcellulose, carbomer polymer, or other gelatinous pharmaceutical agent that is FDA-acceptable. The carrier is preferably water-insoluble, to deter dissolving in water for injection, and may be an oil or a solid—for example paper or other thin medium broadly extended in two dimensions, or a sponge or other medium having generally coarse cellular structure.

### RELATION BACK

This is a continuation-in-part, and claims priority, of U.S. nonprovisional patent application Ser. No. 11/807,355, filed May 25, 2007, which in turn was a continuation and claimed priority of nonprovisional application Ser. No. 11/406,886 filed Apr. 19, 2006—both of which also claimed priority of U.S. provisional patent application Ser. 60/676,865, filed May 2, 2005. The present document accordingly claims priority of all three precursor applications '355, '886 and '865.

### BACKGROUND

Many common substances that are widely used in the U.S. and elsewhere, for legitimate purposes, are unfortunately also frequently diverted for illicit use. In regard to the present invention, of particular interest are substances which are not opiates. One frequent mode of such abuse is snorting. A substance that is solid can be ground mechanically into a fine dust and then snorted, thereby introducing the powder into the nose, where it is rapidly absorbed.

This method of use bypasses the stomach and the liver, resulting in a far more rapid and potent effect of the same amount of substance when ingested by means other than snorting. Snorting nonopiate substances intended to be taken by mouth is dangerous for several reasons.

First, such substances often are sold mixed with other ingredients—designed to improve the easy use of the substance. These can include, according to the manufacturer, various harmless ingredients. Although these ingredients are harmless when taken by mouth, they can have serious health effects when snorted.

Second, delivery of such substances into the system is far more effective. This poses a real risk of harmful effects upon a person's body.

As noted above, some solid substances are easily prepared for snorting by simply grinding them into powder. Among college students this is frequently done by placing a piece of the substance 11 (FIG. 1) in the depression in the bottom of a small bottle 12, and crushing the piece into fragments 11′ with a coin 13.

Often or usually, crushing continues until much of the piece has been reduced to a finer, snortable powder 11″ (FIG. 3). This material is brought near nostrils 15 of the substance abuser, who then inhales to draw a stream 14 of the powder into the nostrils.

Dangerous substances can also be abused by injection. This is accomplished after dissolving the powdered piece in water, aided by heating the water. The resulting mixture of the mechanically produced substance is then injected into the body.

A liquid form of some of the subject substances exists commercially, but it is not formulated to discourage mechanical alteration. The liquid form is relatively much harder than the powder to administer, for legitimate purposes, and therefore its legitimate use is disfavored in some environments. This is particularly true for some such substances (e.g. some substances that are medications), in elementary school—where accurate injections are difficult or impossible. The liquid form also is susceptible to diversion by alteration.

Recently two new products that include such substances have come onto the market. One is a so-called “chewable” product.

Notwithstanding the use of the term “chewable”, the new preparation does no more than merely bulk up the product. A substance abuser can still mechanically pulverize the substance, enabling it to be snorted effectively. It appears that no thought has been given to the common knowledge, among young people, of how easy it is to mechanically alter such substances to allow them to be snorted.

The other substance newly available is in a label-like form which, itself, cannot be pulverized for snorting purposes. For at least two reasons, however, it does not solve the abuse problems described above.

The label-like form can also present cause, in the case of children bearing the product, for taunting and harassment by others. This makes it possible or probable that a child will remove the device to avoid unwanted attention.

Wearing the label-like form under clothing cannot fully eliminate this problem. Junior-high-school and high-school boys typically are required in physical-education classes to undress completely to shower—thus revealing the label-like form and giving rise to the above-mentioned harassing or taunting behavior, perhaps even in aggravated form.

According to government studies, thirty to fifty percent of adolescents in substance-abuse treatment reported so-called recreational use of substances; accordingly such substances were added to a list of concerns. In 2004 it was reported that over five percent of high-school seniors engaged in illegal substance abuse, and as to the college level it has been estimated that as many as one in five college students have illegally practiced substance abuse.

Because legitimate use of many substances is both lawful and common, and the substances are easily available, even the direct discovery of certain substances in routine tests is of little value in identifying abusers. Throughout this document, where the term “substance” appears, to the extent appropriate to the context that term shall be understood to encompass any legally obtained substance susceptible to mechanical alteration for the purpose of snorting or injection.

Thus the threat posed by this type of substance abuse is extremely insidious. Yet it is a known killer.

### SUMMARY OF THE DISCLOSURE

The present invention significantly addresses this problem and provides much needed improvement. In preferred embodiments of a first of its aspects or facets the invention is a method for improving the safety of a nonopiate substance at risk for abuse.

The method includes the step of preparing the substance in a form, other than a label-like form, that tends to deter conversion to powder. It also includes the step of providing the substance for commercial distribution in that form.

The foregoing may represent a description or definition of the first aspect or facet of the invention in its broadest or most general form. Even as couched in these broad terms, however, it can be seen that this facet of the invention importantly advances the art.

In particular, by preparing the substance in a form that tends to deter mechanical conversion to powder, the invention bears significant potential for reducing widespread abuse of common substances available on the market today, as mentioned above. A marked reduction may be expected.

Although the first major aspect of the invention thus significantly advances the art, nevertheless to optimize enjoyment of its benefits preferably the invention is practiced in conjunction with certain additional features or characteristics. Such additional preferences will be discussed shortly.

For the reason explained in the preceding section of this document, a label-like form of the substance (such as is already on the market) cannot effectively prevent the abuse problems mentioned above. Therefore the foregoing definition or description expressly excludes such a label-like form of the substances mentioned above.

Also excluded, but for a very different reason, are substances which are opiates. The reason is that abuse of opiates is addressed by a prior invention and patent to publications of Johannes Bartholomaus and Heinrich Kugelmann—particularly German application 10 2004 032 049.7 of Jul. 1, 2004, and corresponding United States patent application US 2006/0002860 published Jan. 5, 2006.

In particular as to the above-mentioned preferences, preferably the method further includes the step of, before the marketing step, enclosing the substance, in said form, in a small container that a person can harmlessly swallow. Another preference is that this method be applied specifically to a substance that is commonly abused and is available reasonably readily.

Yet another preference is that the mechanical preparing step be performed by dissolving or dispersing the substance into or onto a small object that a person can harmlessly swallow. If this basic preference is observed, then it is further preferable that the object be:
water insoluble—whereby the mechanical preparing for the substance inhibits dissolving the mixture in water for injection; or
a solid—in which case, alternative preferences are that the solid be in the form of paper or other thin medium broadly extended in two dimensions; or in the form of a sponge or other medium having a generally coarse internal mechanical structure.

Other preferred forms are mentioned below.

In preferred embodiments of its second major independent facet or aspect, the invention is a product or composition of matter—in particular, a mechanical preparation of a nonopiate substance that is at risk for abuse, for improving the safety of that substance. The mechanical preparation includes the substance in a form (other than a label-like form) that tends to deter conversion to powder. It also includes an object containing the substance in that form.

The foregoing may represent a description or definition of the second aspect or facet of the invention in its broadest or most general form. Even as couched in these broad terms, however, it can be seen that this facet of the invention importantly advances the art.

In particular, the benefits of this product aspect of the invention are closely related to those of the method aspect discussed above. These benefits particularly include reducing the availability of these substances in conditions that are susceptible to abuse—and thus reducing injuries and deaths caused by such abuse.

Although the second major aspect of the invention thus significantly advances the art, nevertheless to optimize enjoyment of its benefits preferably the invention is practiced in conjunction with certain additional features or characteristics. In particular, preferably the mechanical preparation of the substance can be placed in or on a small object that a person can harmlessly swallow. Other preferences described above for the first aspect of the invention are also applicable here.

In preferred embodiments of its third major independent facet or aspect, the invention is a commercial distribution method for improving the safety of a nonopiate substance at risk for abuse. The method includes the steps of obtaining the substance in a form, other than a label-like form, that tends to deter conversion to powder.

The method also includes the step of selling the substance in such a form. The foregoing may represent a description or definition of the third aspect or facet of the invention in its broadest or most general form.

Even as couched in these broad terms, however, it can be seen that this facet of the invention importantly advances the art. In particular, the sale of commonly abused substances in a pulverization-deterring form enlists the commercial distribution method in the aid of reducing rates and severity of substance abuse.

Although the third major aspect of the invention thus significantly advances the art, nevertheless to optimize enjoyment of its benefits preferably the invention is practiced in conjunction with certain additional features or characteristics. In particular, preferably the selling step includes wholesale selling; it also preferably includes retail selling.

Another preference is that the form include a mixture or suspension of the substance in or on a small object that a person can harmlessly swallow. If this preference is observed, then further preferences include obtaining the small object in the form of a paper, or other thin material broadly extended in two dimensions; or a sponge or other material having generally coarse internal mechanical structure.

The foregoing features and advantages of the invention will be more fully appreciated from the following Detailed Description, considered together with the accompanying drawings and table—of which:

### BRIEF DESCRIPTION OF THE DRAWINGS

FIG. 1 is a perspective or isometric view showing an inverted small bottle with a piece of substance placed thereon, as commonly done in the mechanical alteration of the subject substances for abuse purposes;

FIG. 2 is a like view showing a further stage in the abuse process;

FIG. 3 is another like view showing a final step;

FIG. 4 is a flow chart representing very schematically the steps in practice of my invention; and

FIG. 5 is a benzodiazepine core-structure diagram, with “R” labels denoting common locations of side chains that give these molecules their unique properties.

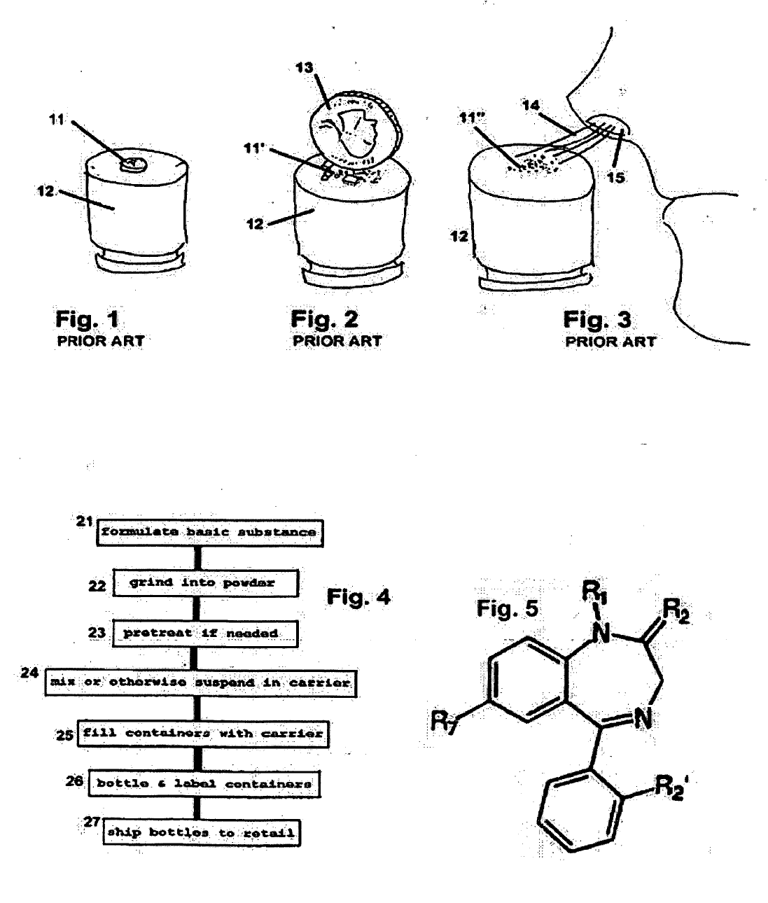

### DETAILED DESCRIPTION OF THE PREFERRED EMBODIMENTS

An objective of this invention, as will be clear from the foregoing discussion, is simply a tendency to deter physical or chemical separation of the substance a from an object that can be harmlessly swallowed. To those skilled in this field, it will also be apparent that it is not absolute prevention that is required—to make practice of my invention extremely valuable to society—but rather only a meaningful tendency to deter.

Absolute deterrence or prevention could be desirable, but achieving that degree of abuse obstruction might be inordinately expensive or complex. Some people intent upon abusing or facilitating abuse of substances may have access to highly advanced physical or chemical separation facilities and abilities, such as are found in industrial or graduate-level laboratories. Extreme measures needed to preclude separation by such individuals may be impractical or produce new adverse consequences.

Rather, the degree of deterrence particularly sought through practice of the present invention is that which obstructs simple home-based methods such as are popular among casual abusers, e.g., as noted above, college students. The very great majority of these people have neither access to special means of separation nor an inclination to expend large sums of money to purchase such services.

Certain substances are widely made available in the U.S. and other countries for addressing various maladies, such as for example attention-deficit disorder. Unfortunately, these substances are frequently diverted for illicit use.

The commonest mode of abuse of such substances is snorting. The substance is ground into a fine dust and then rapidly inhaled through the nose, thereby introducing the substance powder widely onto the nasal membranes where it is rapidly absorbed into the bloodstream.

As noted above, this route of administration bypasses the natural processes of the stomach and liver. As a result, the effect of snorted substances is far more rapid and potent than the effect of the same amount of substance taken by mouth.

Snorting of many substances intended to be taken by mouth is dangerous for several reasons. First, the substances are usually mixed with other ingredients designed to improve the handling characteristics of the substance. These can include, according to the manufacturer, various dyes, conditioning ingredients etc. which may be injurious when snorted.

Second, because of the far more effective delivery of these substances into the body, there is a real risk of harmful results. These may particularly include powerful neurological effects and changes in heart rate and rhythm.

In particular the previously mentioned “chewable” product was not designed to prevent snorting, but rather merely for patients (particularly young children) who are unable to swallow pills intact.

The other new product discussed earlier is a label-like patch. The patch itself is snort proof; however, for at least two reasons it does not solve the abuse problems described above.

First, it does not entirely replace or supplant the common form of the substance, but instead leaves the latter on the market. Second, appearance of the label-like patch makes it obvious to anyone who sees the device that the child or other person uses the above named substance, subjecting the user to taunting, harassment, etc.

As noted above, wearing the label-like form under clothing cannot fully eliminate this problem. In competitive-athletics teams and most physical-education classes, junior-high-school and high-school boys typically are required to undress completely to shower—thus revealing the label-like form and giving rise to the above-mentioned harassing or taunting, perhaps even aggravated by the apparent effort at concealment.

It is possible and I believe likely that a child will remove the label-like article, to avoid such embarrassments—or to sell it to a peer who wants a sensation (“kick”) which the substance may produce.

The government reports that thirty to fifty percent of adolescents in substance treatment reported so-called “illegitimate” use of certain substances (Ritalin®, for example). Official studies were conducted in 2004 and 2005 showing that over five percent of high-school seniors abuse dangerous and harmful substances, and as to the college level estimated that as many as one in five college students have used such substances illegally.

In accordance with preferred embodiments of my invention, legitimate substances meant to ingested by mouth are reformulated into a small object that can be safely swallowed by a person containing the active substance—and whatever other ingredients might be of value in this novel mixture—all prepared as or dissolved in an easy-to-swallow and harmless small object. The nature of the object is chosen to deter snorting and other modes of abuse mentioned above.

Thus the object may comprise a liquid, in which the substance is simply suspended or preferably dissolved; or may comprise a carrier that is a gel, or alternatively a solid. A particularly advantageous liquid is one—such as (merely by way of example) an oil—from which the substance is awkward or difficult to remove without special equipment or training, or preferably both. Liquids from which the substance can be mechanically recovered simply by e.g. drying, evaporation or settling, should not be used.

Although for some purposes possibly it may seem more advantageous to select a carrier of the type mentioned above rather than liquid in general, some particular liquids may be very effective. With respect to certain objects, the substance should be adhered to (as for example by gluing) or embedded within a solid—such as paper, sponge, or a gelatinous etc. confection or pastry—one which is not easy to grind.

The object should be chosen to avoid interfering with the desired legitimate use of the substance, when the object and the substance together are taken by mouth in the approved usual way. This is particularly noteworthy in the case of a solid object such as paper, sponge, etc., since the selected solid must be one that can be chewed, or in any event harmlessly swallowed and digested, notwithstanding its incompatibility with mechanical grinding.

If the substance is placed as small particles throughout or on the object, the combination can be called a two-phase system. If the substance appears to a be completely dissolved in the object, the resulting clear or colored mixture can be termed a one-phase to system.

The most difficult manufacturing problem with a mixture that includes such a carrier substance is ensuring that the substance is distributed evenly, to a reasonable degree, throughout the carrier and substantially without clumping. This may be accomplished by:
smoothly sifting the powdered substance into the rapidly stirred carrier,
making a smooth paste of the substance with a water-miscible agent such as an alcohol or propylene glycol, or
simply using a blender to completely homogenize the mixture of carrier and powder.

Generally these substances, which are commonly used ingredients, are freely soluble in water, and therefore should easily make such a carrier mixture suitable for such combination. The several object ingredients mentioned above include many that are already regulatorily approved; thus the approval requirements for at least initial practice of this invention should be minimal.

I believe that availability of suitable carriers is not a problem, because many gels suitable for use in practicing this invention are inert. Cellulose-based such carriers are routinely used as inert thickeners in various food products.

In any event, the object with the substance in it is placed or molded into an object (e.g. capsule) of, ideally, a commonly used type of appropriate material—or a material with closely related characteristics. Such objects, as is well known, in common use dissolve rapidly to release the substance into the body—and this should be the case in practice of the present invention as well.

Given suitable choice of an object that can be harmlessly swallowed, the resulting combination of the substance and object—enclosed in or molded as a combination—should have essentially the same physiological effects as the common form of the substance.

Snorting of the object contents will, however, be substantially impossible. Physical properties of a carrier such as described above—e.g. a sticky semifluid—prevent breaking the substance into a fine aerosol and thus block its wide mechanical introduction into the nose. Separating substance from object, though possible in a specially equipped lab, would be difficult otherwise.

To make the mixture resistant to injection abuse, the substance should be placed into such a carrier or to other material that is not soluble in water as part of a two-phase system. Given this precaution, the object contents will not be easily converted into injection form.

Thus my invention is advantageously practiced by the steps comprising: mixing 21 (FIG. 4) the basic substance, grinding 22 the mixed substance into powder, applying any pretreatment 23 that may be needed, and mixing or otherwise suspending 24 the substance into a carrier of any of the types mentioned above. Thereafter, the invention may comprise the further steps of filling 25 objects with the substance-containing carrier, bottling and labeling the objects 26, and dispensing the objects for marketing 27.

As set forth in my precursors to the present document, methylphenidate (Ritalin® and others) and amphetamine or amphetamine-like central nervous system stimulant drugs are widely prescribed in the U.S. and other countries for the treatment of attention-deficit disorder. Unfortunately, these drugs are frequently diverted for illicit use.

My precursor documents also teach that methylphenidate for oral administration can be reformulated into a capsule containing the active drug, methylphenidate, and whatever of the inert ingredients that might be of value in this novel formulation, all dispersed or dissolved in a carrier. The carrier may be, among other things, a gel such as methylcellulose, hydroxypropylmethylcellulose, sodium carboxymethylcellulose or a carbomer polymer such as Carbopol® 934P.

In regard to all three main aspects of the invention, substances that are commonly abused and that are targets of the present invention particularly include (1) methyl-based materials such as methylphenidate, particularly Ritalin® (or amphetamine, or other amphetamine-like central nervous system stimulant drugs), and (2) benzodiazepines. Although the invention itself, as indicated by its title, is merely a simple mechanical innovation—not at all depending on sophisticated concepts of molecular biology or the like—these several substances bear very brief mention here.

The Tables below, with text before them and references following, provide an essentially exhaustive enumeration of benzodiazepines, but not all their brand names. I gratefully thank Wikipedia for this tabulation and the accompanying notes below.

### List of Benzodiazepines

From Wikipedia, the free encyclopedia

The below tables contain a list of benzodiazepines core structure shown in FIG. 5—that are commonly prescribed. In the drawing, “R” labels denote common locations of side chains, which give different benzodiazepines their unique properties. The tables also display basic pharmacological characteristics such as half-life and equivalent doses to other benzodiazepines, along with their trade names and primary uses.

The elimination half-life is the time it takes for half of the drug to be eliminated by the body. “Time to peak” refers to when maximum levels of the drug in the blood occur after a given dose. Benzodiazepines generally share the same pharmacological properties, such as anxiolytic, sedative, hypnotic, skeletal muscle relaxant, amnesic and anticonvulsant (hypertension in combination with other antihypertension medications)—important areas of research. Variation in potency of certain effects may of course exist among individual benzodiazepines. Some benzodiazepines produce active metabolites. Active metabolites are produced when a person's body metabolizes the drug into compounds that share a similar pharmacological profile to the parent compound and thus are relevant when calculating how long the pharmacological effects of a drug will last. Long-acting benzodiazepines with long-acting active metabolites such as diazepam and chlordiazepoxide are often prescribed for benzodiazepine or alcohol withdrawal or for anxiety if constant dose levels are required throughout the day. Shorter-acting benzodiazepines are often prescribed for insomnia due to their lesser hangover effect.^{[1] [2] [3] [4] [5]}

### Benzodiazepine Half-Life and Equivalent Dose Table

It is important to note that the elimination half-life of diazepam and chlordiazepoxide as well as other long half-life benzodiazepines is twice as long in the elderly compared to younger individuals. Individuals with an impaired liver also metabolize benzodiazepines more slowly. Many doctors make the mistake of not adjusting benzodiazepine dosage according to age in elderly patients. Thus the equivalent doses below may need to be adjusted accordingly in individuals on short acting benzodiazepines who metabolize long-acting benzodiazepines more slowly and vice versa. The changes are most notable with long acting benzodiazepines as these are prone to significant accumulation in such individuals. For example the equivalent dose of diazepamin an elderly individual on lorazepam may be up to half of what would be expected in a younger individual.^{[6] [7]} Equivalencies between individual benzodiazepines can differ by 20-fold on a milligram-per-milligram basis; awareness of this fact is necessary for the safe and effective use of benzodiazepines.^{[8]}

| | | Time to Peak | Elimination | | |
|---|---|---|---|---|---|
| | Common Brand | (onset of action, | ½-Life (h)† | | |
| Drug Name | Names* | in hours) | [active metabolite] | Therapeutic use | AED‡ |
| Alprazolam | Helex, Xanax, | 1-2 | 6-12 | anxiolytic | 0.5 |
| | Xanor, Onax, | | | | |
| | Alprox, Restyl | | | | |
| Bretazenil | N/A | ? | 2.5 | anxiolytic, | 0.5 |
| | | | | anticonvulsant | |
| Bromazepam | Lectopam, Lexotanil, | 1-3 | 10-20 | anxiolytic | 5-6 |
| | Lexotan, Bromam | | | | |
| Brotizolam | Lendormin, Dormex, | 0.5-2 | 4-5 | hypnotic | 0.25 |
| | Sintonal, Noctilan | | | | |
| Chlordiazepoxide | Librium, Risolid, | 1.5-4 | 5-30 | anxiolytic | 25 |
| | Elenium | | [36-200] | | |
| Cinolazepam | Gerodorm | 0.5-2 | 9 | hypnotic | 40 |
| Clonazepam | Rivotril, Klonopin, | 1-4 | 18-50 | anxiolytic, | 0.5 |
| | Iktorivil, Paxam | | | anticonvulsant | |
| Clorazepate | Tranxene, Tranxilium | variable | 36-100 | anxiolytic, | 15 |
| | | | | anticonvulsant | |
| Clotiazepam | Veratran, Clozan, | 1-3 | 6-18 | anxiolytic | 5-10 |
| | Rize | | | | |
| Cloxazolam | Sepazon, Olcadil | 2-5 (?) | 18-50 | anxiolytic, | 1 |
| | | | | anticonvulsant | |
| Delorazepam | Dadumir | 1-2 | 60-140 | anxiolytic | 1 |
| Diazepam | Antenex, Apaurin, | 1-1.5 | 20-100 | anxiolytic, | 10 |
| | Apzepam, Apozepam, | | [36-200] | anticonvulsant, | |
| | Hexalid, Pax, | | | muscle relaxant | |
| | Stesolid, Stedon, | | | | |
| | Valium, Vival, | | | | |
| | Valaxona | | | | |
| Estazolam | ProSom | 1-5 | 10-24 | hypnotic | 1-2 |
| Etizolam | Etilaam Pasaden, | 1-2 | 6 | anxiolytic, | 1 |
| | Depas | | | hypnotic | |
| Flunitrazepam | Rohypnol, Fluscand | 0.5-3 | 18-26 | hypnotic | 1 |
| | Flunipam, Ronal, | | [36-200] | | |
| | Rohydorm | | | | |
| Flurazepam | Dalmadorm, Dalmane | 1-1.5 | 40-250 | hypnotic | 15-30 |
| Flutoprazepam | Restas | 0.5-9 | 60-90 | hypnotic, | 2-3 |
| | | | | anticonvulsant | |
| Halazepam | Paxipam | 1-3 | 30-100 | anxiolytic | 20-40 |
| Ketazolam | Anxon | 2.5-3 | 30-100 | anxiolytic | 15-30 |
| | | | [36-200] | | |
| Loprazolam | Dormonoct | 0.5-4 | 6-12 | hypnotic | 1-2 |
| Lorazepam | Ativan, Temesta, | 2-4 | 10-20 | anxiolytic, | 1 |
| | Tavor, Lorabenz | | | anticonvulsant | |
| Lormetazepam | Loramet, Noctamid, | 0.5-2 | 10-12 | hypnotic | 1-2 |
| | Pronoctan | | | | |
| Medazepam | Nobrium | ? | 36-200 | anxiolytic | 10 |
| Midazolam | Dormicum, Versed, | 0.5-1 | 3 | hypnotic, | 5-8 |
| | Hypnovel, Dormonid | | (1.8-6) | anticonvulst. | |
| Nimetazepam | Erimin | 0.5-3 | 14-30 | hypnotic | 5 |
| Nitrazepam | Mogadon, Alodorm, | 0.5-7 | 15-38 | hypnotic, | 10 |
| | Pacisyn, Dumolid | | | anticonvulsant | |
| Nordazepam | Madar, Stilny | ? | 50-120 | anxiolytic | 10 |
| Oxazepam | Serenid, Serepax, | 3-4 | 4-15 | anxiolytic | 20 |
| | Oxabenz, Oxapax | | | | |
| Phenazepam | Phenazepam | 1.5-4 | 60 | anxiolytic, | 1 |
| | | | | anticonvulsant | |
| Pinazepam | Domar | ? | 40-100 | anxiolytic | 20 |
| Prazepam | Lysanxia, Centrax | 2-6 | 36-200 | anxiolytic | 20 |
| Premazepam | N/A | 2-6 | 10-13 | anxiolytic | 3.75 |
| Quazepam | Doral | 1-5 | 39-120 | hypnotic | 20 |
| Temazepam | Restoril, Normison, | 0.5-3 | 8-22 | hypnotic | 20 |
| | Euhypnos, Temaze, | | | | |
| | Tenox | | | | |
| Tetrazepam | Mylostan | 1-3 | 3-26 | skeletal muscle | 100 |
| | | | | relaxant | |
| Triazolam | Halcion, Rilamir | 0.5-2 | 2 | hypnotic | 0.25 |

### Atypical Benzodiazepine Receptor Ligands

| | Common | Elimination | | |
|---|---|---|---|---|
| | Brand | ½-Life (hr)^{†} | Primary | |
| Drug Name | Names* | (active metabolyte) | Effects | AED^{‡} |
| Clobazam | Frisium, | 8-60 | anxiolytic, | 20 |
| | Urbanol | | convulsant | |
| DMCM | ? | ? | anxiogenic | N/A |
| Flumazenil° | Anexate, | 1 | antidote | typ. dose |
| | Lanexat, | | | 0.2-0.6° |
| | Mazicon, | | | |
| | Romazicon | | | |
| Eszopiclone^{§} | Lunesta | 6 | hypnotic | 3 |

| | Common | Elimination | | |
|---|---|---|---|---|
| | Brand | Half-Life (hr)^{†} | Primary | |
| Drug Name | Names* | (active metabolyte) | Effects | AED^{‡} |
| Zaleplon^{§} | Sonata | 1 | hypnotic | 20 |
| | Starnoc | | | |
| Zolpidem^{§} | Ambien, | 2.6 | hypnotic | 20 |
| | Nytamel, | | | |
| | Stilnoct, | | | |
| | Stilnox, | | | |
| | Zoldem, | | | |
| | Zolnod | | | |
| Zopiclone^{§} | Imovane, | 4-6 | hypnotic | 15 |
| | Rhovane, | | | |
| | Ximovan; | | | |
| | Zileze; | | | |
| | Zimoclone; | | | |
| | Zimovane; | | | |
| | Zopitan; | | | |
| | Zorclone | | | |

*Not all trade names are listed. Click on drug name [in the Wikipedia website] to see a more comprehensive list.
^{†}The duration of apparent action is usually considerably less than the half-life. With most benzodiazepines, noticeable effects usually wear off within a few hours. Nevertheless, as long as the drug is present it will exert subtle effects within the body. These effects may become apparent during continued use or may appear as withdrawal symptoms when dosage is reduced or the drug is stopped.
^{‡}“AED” (“Approximate Equivalent Dose”) is shown in milligrams (mg), and based on clinical experience - but may vary between individuals. [1] (http://www.benzo.org.uk/bzequiv.htm)
^{§}The molecular structure of these drugs differs from the benzodiazepine molecule but they work on benzodiazepine receptors with the same or similar effects and are cross-tolerant drugs.
°Flumazenil is given to reverse the effects of benzodiazepines and similar drugs, and dosage range listed will vary depending on which drug is being counteracted, what dosage the first drug was given in, and whether the flumazenil is given actually to reverse overdose or just to reduce side effects.

### REFERENCES

1. ̂ Golombok S. Lader M (August 1984). “The psychoparmacological effects of premazepam, diazepam and placebo in healthy human subjects” (http;//www.pubmedcentra.nih.gov/articlerender.fegi?tool=pmcentrez&artid=1463527). Br J Clin Pharmacol 18 (2): 127-33. PMC 1463527 (http://www.pubmedcentral.gov/articlerender.fcgi?tool-pmcentrez&artid=1463527). PMID 6148956 (http://www.ncbi.nlm.nih.gov/pubmed/6148956). http://www.pubmedcentral.nih.gov/articlerender.fcgi?tool=pmcentrez&artid=1463527.
2. ̂ de Visser S J, van der Posts J P, de Waal P P, Cornet F, Cohen A F, van Gerven J M (January 2003). “Biomarkers for the effects of Benzodiazepines in healthy volunteers” (http://www3.interscience.wiley.com/cgi-bin/fulltext/1188-82375/PDFSTART?CRETRY=1&SRETRY=0) (PDF). Br J Clin Pharmacol 55 (1):39-50. doi:10.1046/j.1365-2125.2002.t01-10-01714.x (http://dx.doi.org/10.1046%2Fj.1365-2125.2002.t01-10-10714.x). PMC 1884188 (http://www.pubmedcentral.gov/articlerender.fcgi?tool=pmcentrez&artid=1884188). PM1D 12534639 (http://www.ncbi.nlm.nih.gov/pubmed/12534639). http://www.ncbi.nlm.nih.gov/pubmed/12534639). http://www3.interscience.wiley.com/cgi-bin/fulltext/118882375/PDFSTART?CRETRY=1&SRETRY=0.
3. ̂ “Benzodiazepine Names” (http://www.non-benzodiazepines.org.uk/benzodiazepine-names.html), non-benzodiazepines.org.uk. http:/www.non-benzodiazepines.org.uk/benzodiazepine-names.html. Retrieved 2009-04-05.
4. ̂ C. Heather Ashton (March 2007), “Benzodiazepine Equivalence Table” (http://www.benzo.org.uk/bzequiv.htm). benzo.org.uk. http://www.benzo.org.uk/bzequiv.htm. Retrieved 2009-04-05.
5. ̂ Bob, Dr (July 1995), “Benzodiazepine Equivalence Charts” http://www.dr-bob.org/tips/bzd.html). dr-bob.org. http://www.dr-bob.org/tips/bzd.html. Retrieved 2009-04-05.
6. ̂ Salzman, Carl (15 May 2004). Clinical geriatric psychopharmacology (http://www.books.google.com/?id-RXvpjJ1Un2gC) (4th ed.). USA: Lippincott Williams & Wilkins. pp. 450-453. ISBN 978-0781743808. http://books.google.com/?id=RXvpjJlUn2gC.
7. ̂ Delco F, Tchambaz L., Schlienger R, Drewe J, Krähenbühl S (2005), “Dose adjustment in patients with liver disease”. Drug Saf 28 (6): 529-45. doi:10.2165/00002018-200528060-00005 (http://dx.doi.org/10.2165%2F00002018-200528060-00005). PMID 15924505 (http://www.ncbi.nlm.nih.gov/pubmed/15924505).
8. ̂ Riss, J.; Cloyd, J.; Gates, J.; Collins, S. (August 2008). “Benzodiazepines in epilepsy: pharmacology and pharmacokinetics.”. Acta Neurol Scand 118 (2): 69-86. doi:10.1111/j.1600-0404.2008.01004.x (http://dx.doi.org/10.1111%2Fj.1600-0404.2008.01004.x). PMID 18384456 (http://www.ncbi.nlm.nih.gov/pubmed/18384456).
9. ̂ van Steveninck A L et al. (1996). “Pharmacokinetic and pharmacodynamic interactions of bretazenil and diazepam with alcohol.” (http://www.pubmedcentral.nih.gov/articlerender.fcgi?tool=pmcentrez&ftid=2042631). British Journal of Clinical Pharmacology 41 (6): 565-573. doi:10.1046/j.1365-2125.1996.38514.x (http:/dx.doi.org/10.1046%2Fj.1365-2125.1996.38514.x). PMC 2042631 (http://www.pubmedcentral.gov/articlerender.fcgi?tool=pmcentrez&artid=2042631). PMID 8799523 (http://www.ncbi.nlm.nih.gov/pubmed/879523). http://www.pubmedcenttral.nih.gov/articlerender.fcgi?tool=pmcentrez&artid=2042631.
0. ̂ Sostmann H J, Sostmann H, Crevoisier C, Bircher J (1989). “Dose equivalence of midazolam and triazolam. A psychometric study based on flicker sensitivity, reaction time and digit, symbol substitution test”. Eur. J. Clin. Pharmacol. 36 (2): 181-7. PMID 2721543 (http://www.ncbi.nlm.nih.giv/pubmed/2721543).

The foregoing disclosure is intended to be merely exemplary and not to limit the scope of the invention—which is defined by the appended claims.

I claim:

1. A method for improving the safety of a nonopiate substance at risk for abuse; said method comprising the steps of:
formulating the substance into a form, other than a label-like form, that tends to deter conversion to powder; and
providing the nonopiate substance for commercial distribution in said form.

2. The method of claim 1, further comprising the step of:
before the providing step, placing the substance, in said form, in a condition such that the substance can be easily and harmlessly swallowed by a person.

3. The method of claim 1, wherein:
the formulating step is performed by dissolving or dispersing the substance into or onto a carrier that can be easily and harmlessly swallowed by a person.

4. The method of claim 3, wherein:
the carrier is water-insoluble;
whereby the mixture inhibits dissolving the substance in water for injection.

5. The method of claim 3, wherein:
the carrier is a solid.

6. The method of claim 5, wherein:
the solid is in the form of paper or other thin medium broadly extended in two dimensions but substantially no more than two dimensions.

7. The method of claim 5, wherein:
the solid is in the form of a sponge or other medium having a generally coarse cellular structure.

8. The method of claim 1, wherein:
the nonopiate substance is methylphenidate or amphetamine, or other amphetamine-like central nervous system stimulant drug.

9. The method of claim 1, wherein:
the nonopiate substance is benzodiazepine.

10. A substance mixture for improving the safety of a nonopiate substance at risk for abuse; said mixture comprising:
the nonopiate substance, in a form, other than a label-like form, that tends to deter conversion to powder; and
an object containing the nonopiate substance in said form.

11. The substance mixture of claim 10, wherein:
said form comprises a mixture of the nonopiate substance with at least one other substance, in or on said object;
wherein said mixture can be harmlessly swallowed by a person.

12. The substance mixture of claim 10, wherein:
the nonopiate substance is methylphenidate or amphetamine, or another amphetamine-like central nervous system stimulant drug.

13. The substance mixture of claim 10, wherein:
the nonopiate substance is benzodiazepine.

14. A commercial distribution method for improving the safety of a nonopiate substance at risk for abuse; said method comprising the steps of:
obtaining the nonopiate substance in a form, other than a label-like form, that tends to deter conversion to powder; and
selling the nonopiate substance in said form.

15. The method of claim 14, wherein:
the selling step comprises wholesale selling.

16. The method of claim 14, wherein:
the selling step comprises retail selling.

17. The method of claim 14, wherein:
said form comprises a mixture of the nonopiate substance with at least one other substance, in or on an object that can be harmlessly swallowed by a person.

18. The method of claim 17, wherein:
the object is in the form of a paper, or other thin material broadly extended in two dimensions but substantially no more than two dimensions; or a sponge or other material having generally coarse cellular structure.

19. The method of claim 14, wherein:
the nonopiate substance is methylphenidate or amphetamine, or other amphetamine-like central nervous system stimulant drug.

20. A method for improving the safety of a substance at risk for abuse; said method comprising the steps of:
formulating the substance into a form, other than a label-like form, that tends to deter conversion to powder;
then placing the substance, in said form, in a condition such that the substance can be easily and harmlessly chewed and swallowed by a person; and
then providing the substance for commercial distribution in said form.

21. The method of claim 20, wherein:
the formulating step is performed by dissolving or dispersing the substance into or onto a carrier that can be easily and harmlessly chewed and swallowed by a person.

22. The method of claim 21, wherein:
the carrier is water-insoluble;
whereby the mixture inhibits dissolving the substance in water for injection.

23. The method of claim 22, wherein:
the carrier is a gel.

24. The method of claim 23, wherein:
the carrier gel is methylcellulose, or hydroxypropylmethylcellulose, or sodium carboxymethylcellulose, or Carbopol® 934P, or another carbomer polymer.

25. The method of claim 20, wherein:
the substance is a nonopiate, namely methylphenidate or amphetamine, or another amphetamine-like central nervous system stimulant drug.

26. The method of claim 21, wherein:
the carrier is a solid.

27. The method of claim 26, wherein:
the solid is in the form of paper or other thin medium broadly extended in two dimensions but substantially no more than two dimensions.

28. The method of claim 26, wherein:
the solid is in the form of a sponge or other medium having a generally coarse cellular structure.

29. The method of claim 20, wherein:
the nonopiate substance is benzodiazepine.
